(19) United States
(12) Patent Application Publication
(10) Pub. No.: US 2020/0230115 A1
(43) Pub. Date: Jul. 23, 2020

(54) METHODS OF TREATING INFLUENZA-ASSOCIATED VIRAL PNEUMONIA

(71) Applicant: University of Cincinnati, Cincinnati, OH (US)

(72) Inventors: Francis X. McCormack, Cincinnati, OH (US); Nikolaos Nikolaidis, Cincinnati, OH (US)

(21) Appl. No.: 16/633,242

(22) PCT Filed: Jul. 24, 2018
(86) PCT No.: PCT/US18/43406

§ 371 Date: Jan. 23, 2020

Related U.S. Application Data
(60) Provisional application No. 62/535,988, filed on Jul. 24, 2017.

(51) Int. Cl. A61K 31/436; A61K 9/00; A61P 31/16

(52) U.S. Cl. CPC A61K 31/436; A61P 31/16; A61K 9/0053; A61K 9/0075

(57) ABSTRACT

A method of reducing risk of influenza A virus infection progressing to viral pneumonia in a patient diagnosed with or suspected of having influenza A virus infection is provided herein, the method comprising administering to the patient an effective amount of an inhibitor of mammalian target of rapamycin (mTOR). Also provided are a method of reducing severity of influenza A virus infection and a method of treating a patient diagnosed with or suspected of having influenza A virus infection, the method comprising administering to the patient via inhalation an effective amount of an mTOR inhibitor, such as rapamycin.

### CROSS REFERENCE TO RELATED APPLICATION

This application claims priority to U.S. Provisional Application Ser. No. 62/535,988, filed Jul. 24, 2017, which application is hereby incorporated by reference in its entirety.

### TECHNICAL FIELD

This disclosure relates to the field of viral pneumonia therapy. More specifically, this disclosure relates to therapeutic methods of treating influenza A virus infection and reducing the risk of developing pneumonia associated with influenza A virus infection by administering one or more inhibitors of the mammalian target of rapamycin (mTOR).

### BACKGROUND

Influenza virus spreads across the globe in seasonal epidemics that cause 3-5 million cases of severe illness and claim up to 500,000 lives each year. In most patients, influenza symptoms last 7-10 days and are limited to fever, rhinitis, pharyngitis, myalgia, headache, and cough resulting from viral bronchitis. Although individual risk for mortality is low, a significant subset of infected patients develops pneumonia, the most lethal consequence of influenza. Those at the highest risk for infection and complications include pregnant women, the elderly (>65 years of age), the very young (<5 years of age, and especially less than 2 years of age), smokers, those exposed to inhaled toxicants such as oxygen and air pollutants, and patients with chronic illnesses such as diabetes and emphysema. Increased risk is often ascribed to impairment of the immune system and differential susceptibility of the airway epithelium is seldom considered in mechanistic studies of host factors that predispose to influenza A infection and poor outcomes.

On inhalation into the respiratory tract, influenza virus affects the nasal, oropharyngeal, and upper airway epithelium. Receptors at the apex of abundant trimeric hemagglutinin (HA) proteins jutting from the viral envelope bind to terminal sialic acid residues on glycoproteins and glycolipids of host cells, promoting internalization of the virions by endocytosis. Proteolytic cleavage of the HA by soluble and membrane-bound enzymes facilitates a key conformational change that mediates fusion of the viral and endosomal envelopes. This process is aided by viral pore (M2) proteins, which facilitate endosomal acidification and drive the release of core proteins and viral RNA into the cytoplasm, which are then transported into the nucleus. There, host replication machinery is coopted to produce viral RNA and protein that are packaged into new viral particles and transported to the cell surface. Neuraminidase cleaves the sialic acid link between the viral HA and cell surface glycans that tether the virus to the host cell, liberating viral progeny to perpetuate the infectious cycle.

Development of pneumonia is the most lethal consequence of influenza, increasing mortality more than 50-fold compared with uncomplicated infection. The spread of influenza A virus from the epithelium of the conducting airway to the alveolar epithelium is a pivotal event in the pathogenesis of primary viral pneumonia. Host susceptibility to influenza A virus pneumonia is often attributed to altered immunity; cell autonomous vulnerability states of the alveolar epithelium, such as proliferative tone, are rarely considered.

A need exists for greater understanding of the epithelial factors that promote the spread of influenza from the conducting airway to the alveolar compartment, leading to progression from bronchitis to pneumonia. Further, a need exists for the development of new strategies and therapies to reduce the severity of influenza infection and/or reduce the risk of developing pneumonia as a result of influenza A infection.

### SUMMARY

The present disclosure demonstrates that mitogenic stimulation of alveolar epithelial type II cells renders them susceptible to influenza A virus (IAV) infection in an mTOR-dependent manner. Accordingly, provided herein are methods of treating and/or ameliorating influenza A virus infection in patients by administering mTOR inhibitors.

In one embodiment, a method of reducing risk of influenza A virus infection progressing to viral pneumonia in a patient diagnosed with or suspected of having influenza A virus infection is provided, the method comprising administering to the patient an effective amount of an mTOR inhibitor.

In another embodiment, a method of reducing severity of influenza A virus infection, the method comprising administering to a patient suffering from influenza A virus infection an effective amount of a pharmaceutical aerosol comprising an mTOR inhibitor.

In another embodiment, a method of treating a patient diagnosed with or suspected of having influenza A virus infection, the method comprising administering to the patient via inhalation an effective amount of rapamycin, whereby the administering reduces risk of the patient developing viral pneumonia.

These and other objects, features, embodiments, and advantages will become apparent to those of ordinary skill in the art from a reading of the following detailed description and the appended claims.

### BRIEF DESCRIPTION OF THE DRAWINGS

FIG. 1. KGF administration enhances IAV-induced mortality and viral burden. (A) Mice were given i.t. IAV (1,500 viral particles) in PBS with or without 5 mg/kg KGF, and vital status was monitored over the course of 11 d (n=4/group; P=0.01 for difference between PBS and KGF groups). (B) IAV (1,500 viral particles) in PBS containing 100, 10, 1, 0.1, or 0 μg KGF were administered to mice by the i.t. route, and vital status was monitored over the course of 11 d (n=4/group; P<0.01 for KGF 100 or 10 mg/kg vs. PBS control). (C) Mice were treated with i.t. PBS alone (black line) or PBS containing KGF (5 mg/kg) at time 0 (gray line) or 120 h (gray dashed line) before infection with IAV. Vital status was monitored for 10 d postinfection (n=4/group; *P<0.05 for −120 h and 0 h groups). (D) PBS with or without IAV (1,500 viral particles) and with or without 5 mg/kg KGF was administered i.t., and viral particles were quantified in lung homogenates by real-time PCR at 0, 24, 48, and 72 h postinfection. **P<0.01.

FIG. 2. Intrapulmonary KGF treatment increases IAV-induced proinflammatory cytokine levels. PBS with or without IAV (1,500 viral particles) and with or without 5 mg/kg KGF was administered i.t., and inflammatory cytokines were measured. (A and B) Multiplex analysis of 43 cytokines and chemokines was conducted 72 h later in acellular BALF and serum, respectively. Data in (A) and (B) were presented for most informative cytokines as fold change from PBS control (n=3 mice/group). (C-F) IL-6, TNFα, KC (CXCL1), and MCP-1 (CCL2) levels were measured by ELISA in whole-lung homogenates at 0, 24, 48, and 72 h postinfection. ***P<0.001; **P<0.01.

FIG. 3. IAV (1,500 viral particles) was administered in PBS with or without 5 mg/kg KGF. Lungs were inflation fixed and harvested at 0, 48, or 120 h. (A and B) Immunohistochemical staining for IAV-NP was performed on lung sections (bars, 200 μm). (C-E) IAV-NP staining of the 48-h lung sections was scored on a semiquantitative scale in a blinded manner. ***P<0.001; *P<0.05.

FIG. 4. KGF enhances AECII proliferation and susceptibility to IAV infection. Mice were treated with i.t. KGF (5 mg/kg) 0, 24, 48, or 120 h before IAV infection. Twenty-four hours after challenge with IAV, lungs were harvested and dissociated and dispersed-lung cells were stained with antibody to pro SP-C, IAV-NP, and Ki67 and analyzed by flow cytometry. (A) Experimental timeline for (B-D) is shown. (B) Percentage of pro SP-C-positive cells that also expressed the proliferation marker Ki67 was determined. (C) Percentage of pro SP-C-positive cells that were infected with IAV was determined by measuring IAV-NP staining. (D) Mean channel fluorescence (MCF) of IAV-NP staining in pro SP-C-positive cells are shown (n=4/group). **P<0.01; ***P<0.001.

FIG. 5. KGF enhances AECII proliferation and susceptibility to IAV infection, ex vivo. AECII were isolated from mice that were treated with i.t. KGF (5 mg/kg) 24, 48, or 120 h before AECII isolation, incubated with IAV in media or media alone ex vivo for 18 h, fixed and stained with antibodies to Ki67, pro SP-C, and IAV-NP, and analyzed by flow cytometry. (A) Experimental timeline for (B-D) is shown. (B) Percentage of pro SP-C-positive cells that also expressed Ki67 was determined. (C) Percentage of pro SP-C-positive cells that stained with IAV-NP are shown. (D) MCF of IAV-NP in pro SP-C-positive cells is shown. (E) Two-parameter histogram of Ki67 vs. IAV-NP staining of AECII isolated from mice pretreated with KGF 48 h before isolation and infected ex vivo is shown. Cells were harvested and analyzed by flow cytometry at 24 h after infection. Representative IAV-NP staining of Ki67-positive AECII (upper histogram) and Ki67-negative AECII (lower histogram) is shown. IAV-NP mean channel MCF data are shown for one of two independent experiments (n=4). (F) Western blot of AECII isolated from mice that were treated with i.t. KGF (5 mg/kg) 24, 48, 120 h before AECII isolation and incubated with IAV in media or media alone ex vivo for 18 h before harvesting. Blots were stained with antibodies to IAV M1 and to GAPDH as a loading control. (G) Densitometry analysis of AECII Western blot is shown. *P<0.05; ***P<0.001.

FIG. 6. Rapamycin inhibits KGF-induced mortality, proliferation, cytokine expression, and susceptibility to IAV infection, in vivo. Mice were pretreated with i.p. rapamycin for 4 d before i.t. administration of PBS alone or PBS containing KGF (5 mg/kg) with and without IAV. (A) Twenty-four hours after inoculation, immunohistochemical staining for phospho-S6 was performed on lung sections and scored using a semiquantitative scale in a blinded manner. (B) Forty-eight hours after challenge with KGF, lungs were harvested and dissociated, and dispersed lung cells were stained with antibodies to pro SP-C, phospho-S6, and Ki67 and examined by flow cytometry to determine the percentage of pro SP-C+Ki67-positive cells. (C) Seventy-two hours after challenge with KGF, lungs were harvested and dissociated, and dispersed lung cells were stained with antibody to pro SP-C and IAV-NP and examined by flow cytometry to determine the percentage of pro SP-C+IAV-NP positive cells. (D) Vital status was monitored over the course of 9 d after IAV infection of rapamycin-pretreated mice and controls (P<0.05 for KGF/IAV vs. KGF/IAV/rapa). (E) IL-6 levels were measured by ELISA in whole-lung homogenates at 48 h after infection. (F) Western blotting was performed on whole-lung homogenates isolated 24 h after IAV infection. Blots were stained with antibodies to IAV-NP and to actin as a loading control. ***P<0.001; **P<0.01; *P<0.05.

FIG. 7. Intrapulmonary KGF treatment increases IAV-induced proinflammatory cytokine levels. IAV (1,500 viral particles) in PBS with or without 5 mg/kg KGF was administered i.t., and inflammatory cytokines were measured. Multiplex analysis of 18 of the most informative cytokines and chemokines of 43 tested was conducted 24 and 72 h later in acellular BALF (open bars) and serum (closed bars): (A) Miip-1β; (B) KC; (C) Eotaxin; (D) RANTES; (E) TARC; (F) IFN γ; (G) Miip-1α; (H) MIP-2; (I) IL-4; (J) IP-10; (K) MCP-5; (L) TNF-α; (M) MIG; (N) IL-6; (O) MCP-1; (P) G-CSF; (Q) M-CSF; and (R) LIF.

FIG. 8. Susceptibility of rat AECII in primary culture to IAV infection is reduced by rapamycin (mTOR inhibitor) or BGJ398 (FGFR inhibitor). AECII were isolated from Sprague-Dawley rats and grown on collagen-IV coated tissue culture plates. (A) Light micrograph of typical morphology of AECII cells after 4 d of incubation in culture. (B) Lysotracker-labeled monolayers demonstrating prominent lamellar bodies associated with AECII. (C) Western blot of KGF (50 ng/mL) induced increases in PI3K/mTOR signaling in rat AECII. (D) Western blot of AECII cell lysates 18 h after IAV infection is conducted in the presence or absence of KGF (50 ng/mL) in cells and pretreatment with FGFR pan-inhibitor 2.5 μM BGJ398 or the mTOR inhibitor 40 nM rapamycin. Blots were stained with antibodies to IAV M1 and to actin as a loading control.

### DETAILED DESCRIPTION

The details of one or more embodiments of the presently-disclosed subject matter are set forth in this document. Modifications to embodiments described in this document, and other embodiments, will be evident to those of ordinary skill in the art after a study of the information provided herein.

While the following terms are believed to be well understood by one of ordinary skill in the art, definitions are set forth to facilitate explanation of the presently-disclosed subject matter.

Unless defined otherwise, all technical and scientific terms used herein have the same meaning as commonly understood by one of ordinary skill in the art to which the presently-disclosed subject matter belongs.

Unless otherwise indicated, all numbers expressing quantities of ingredients, properties such as reaction conditions, and so forth used in the specification and claims are to be understood as being modified in all instances by the term “about.” Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, pH, size, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

An “effective amount,” as used herein, refers to an amount of a substance (e.g., a therapeutic compound and/or composition) that elicits a desired biological response. In some embodiments, an effective amount of a substance is an amount that is sufficient, when administered to a subject suffering from or susceptible to a disease, disorder, and/or condition, to treat, diagnose, prevent, and/or delay and/or alleviate one or more symptoms of the disease, disorder, and/or condition. As will be appreciated by those of ordinary skill in this art, the effective amount of a substance may vary depending on such factors as the desired biological endpoint, the substance to be delivered, the target cell or tissue, etc. For example, the effective amount of a formulation to treat a disease, disorder, and/or condition is the amount that alleviates, ameliorates, relieves, inhibits, prevents, delays onset of; reduces severity of and/or reduces incidence of one or more symptoms or features of the disease, disorder, and/or condition. Furthermore, an effective amount may be administered via a single dose or via multiple doses within a treatment regimen. In some embodiments, individual doses or compositions are considered to contain an effective amount when they contain an amount effective as a dose in the context of a treatment regimen. Those of ordinary skill in the art will appreciate that a dose or amount may be considered to be effective if it is or has been demonstrated to show statistically significant effectiveness when administered to a population of patients; a particular result need not be achieved in a particular individual patient in order for an amount to be considered to be effective as described herein.

Influenza A viruses (IAVs) are negative-sense, single-stranded, segmented RNA viruses. IAVs are categorized into subtypes according to hemagglutinin (H) and neuraminidase (N) protein types on the surface of the viral envelope. There are 18 known H antigens (H1 to H18) and 11 known N antigens (N1 to N11). Each subtype has mutated into a variety of strains having different pathogenic profiles. At present, H1N1 and H3N2 are the known subtypes spread widely among humans. Influenza A infection symptoms include fever, chills, cough, sore throat, runny or stuffy nose, muscle and body aches, headache, and fatigue. Severe influenza infection may lead to further complications, including bronchitis, pneumonia, and sinus and ear infections.

The term “administering,” as used herein, refers to any route of administering an effective amount of a therapeutic agent. In some embodiments, the administering includes, but is not limited to, oral, intravenous, subcutaneous, intramuscular, intraperitoneal, sublingual, rectal, nasal, pulmonary or inhaled, and transdermal administration. In specific embodiments, the therapeutic agent is administered orally or via inhalation. In a very specific embodiment, the therapeutic agent is administered via inhalation.

As used herein, the term “mTOR inhibitor” refers to an agent that can reduce the expression level and/or activity of mTOR protein and/or mRNA. In some embodiments, an mTOR inhibitor can reduce the expression level of mTOR mRNA. In some embodiments, an mTOR inhibitor can reduce the expression level of mTOR polypeptide. In some embodiments, an mTOR inhibitor can reduce the activity of mTOR polypeptide. As used herein, the term “mTOR” refers to a serine/threonine kinase of the PI3K enzyme family that functions as the catalytic subunit of the mTORC1 and mTORC2 complexes (e.g. NCBI Gene ID: 2475). The sequences of mTOR nucleic acids and polypeptides in a number of species are known in the art (e.g. human mTOR nucleic acid, NCBI Ref Seq: NM_004958; and human mTOR polypeptides, NCBI Ref Seq: NP_004949). mTOR is also referred to as FRAP, RAFT 1, and RAPT. mTOR inhibitors can inhibit mTOR via any known mechanism, including, e.g., binding of a competitive inhibitor, binding of a non-competitive inhibitor, increasing the rate of degradation of mTOR polypeptides, blocking the biosynthesis, transcription, and/or translation of mTOR, blocking the targeting of AKT to mTOR, and increasing the inhibition of mTOR by TSC1/2. mTOR inhibition can be determined by methods well known in the art, e.g. by detecting the level of phosphorylation of mTOR targets such as p70-S6 kinase 1 (S6K1), 4EBP1, and Akt, where inhibition or a decreased in the phosphorylation of these targets indicates effective inhibition of mTOR. In certain embodiments, an agent can increase or decrease the expression of a component of the targeted signaling pathway. Components of the mTOR signaling pathway include, but are not limited to RAPTOR, DEPTOR, Rheb, AKT, RICTOR, GPL, and HIF-1. The mTOR signaling pathways have been described in the art, e.g. in Dunlop and Tee, Cell Signal 21(6):827-35 (2009); Laplante and Sabatini, J Cell Sci 122(Pt20):3589-94 (2009); Kudchodkar, et al. PNAS 103(38):14182-7 (2006); which are incorporated by reference herein in their entireties.

Non-limiting examples of mTOR inhibitors for use in the methods described herein include, but are not limited to, one or more of rapamycin, everolimus, temsirolimus, deforolimus, zotarolimus, biolimus (umirolimus), and others disclosed, for example, in U.S. Pub. No. 20120064134 and U.S. Pat. No. 9,737,514, each of which is incorporated by reference in its entirety. In a specific embodiment, an mTOR inhibitor is selected from the group consisting of rapamycin, everolimus, temsirolimus, deforolimus, and combinations thereof. In a specific embodiment, the mTOR inhibitor is rapamycin.

A patient “suspected of having” influenza A infection refers to a patient who exhibits one or more clinical symptoms consistent with influenza A virus infection. A patient suspected of having influenza A infection also includes a patient who has been exposed to influenza A virus but does not yet exhibit one or more clinical symptoms.

The terms “treat,” “treatment,” and “treating,” as used herein, refer to a method of alleviating or abrogating a disease, disorder, and/or symptoms thereof in a subject, including a mammal. In certain embodiments, the subject is a human patient.

“Reducing severity” refers to ameliorating or lessening severity of a viral infection or one or more symptoms thereof. “Reducing severity” also includes shortening or reduction in duration of one or more symptoms of infection.

The presently disclosed studies were initially designed to examine the therapeutic potential of KGF, a potent pulmonary epithelial mitogen that is known to promote expression of innate immune proteins with anti-IAV actions, including the collectins SP-A and SP-D. Surprisingly, it was found that intrapulmonary instillation of KGF in mice worsened IAV-induced mortality, increased pulmonary viral load and inflammatory tone, and accelerated the spread of IAV from the conducting airways to the alveolar compartment. KGF also directly enhanced in vivo susceptibility of AECII to IAV infection in a manner that correlated with their proliferative state. The finding that AECII isolated from KGF-pretreated mice were also more permissive to ex vivo IAV infection suggests that cell autonomous mechanisms, rather than a more global effect on host immunity, are a key driver of AECII infectious susceptibility. These data suggest that the mitogenic tone of AECII in the lung is a potential host risk factor to be considered in the approach to prophylaxis and clinical management of IAV and may be a potential therapeutic target for IAV pneumonia.

Influenza infects airway epithelial cells and Clara cells of the distal conducting airway. At the level of the alveolus, alveolar macrophages, AECI and AECII cells can all be infected with IAV, but among these, AECII are selectively targeted and uniquely capable of sustaining a productive infection. IAV infection of the pulmonary parenchyma results in loss of expression of SP-C, consistent with reduction in viability and differentiated functions of AECII cells. This consequence of IAV infection critically affects alveolar homeostasis through loss of AECII roles in regulating innate immune responses, lowering surface pressures through surfactant synthesis and secretion, and disruption of lumenal fluid and electrolyte balance. Pathological evaluations of lung samples from cases of fatal influenza uniformly reveal evidence for diffuse alveolar damage, consistent with direct injury to the alveolar epithelium as a mechanism of death in influenza pneumonia.

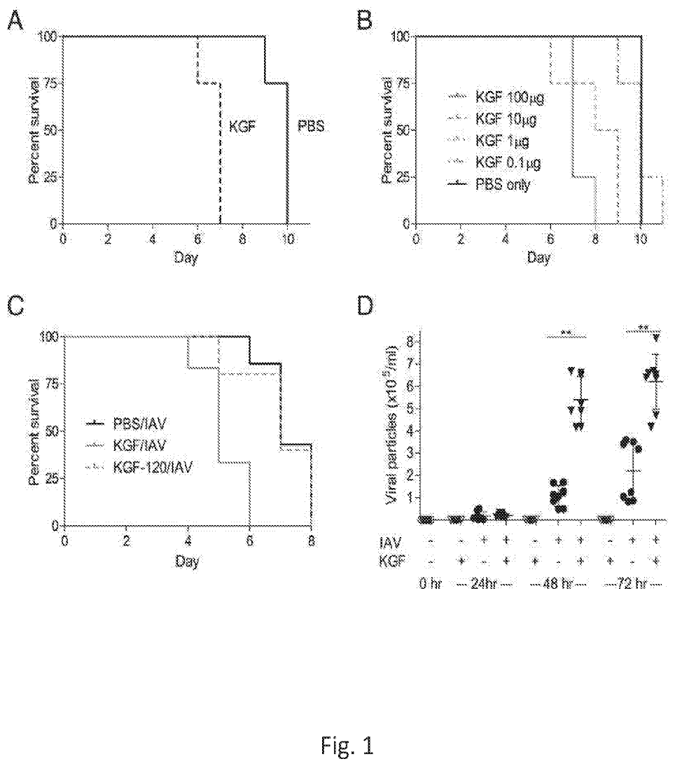

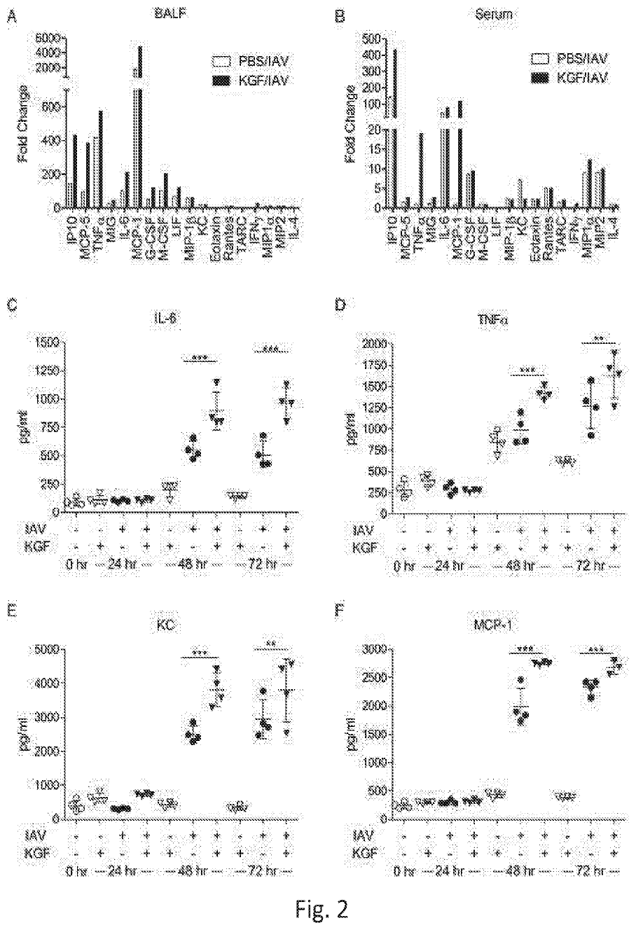

The spread of IAV from the epithelium of the conducting airway to the alveolar epithelium is therefore a pivotal event in the pathogenesis of fatal influenza pneumonia. Multiple host factors align to impede this transition, including physical defenses such as mucociliary clearance, humoral defenses including antibodies, cellular defenses such as natural killer cells, and inhibition of proteolytic activation of the virus. To explore the mechanism of the KGF effect, the possibility that the mitogen was enhancing mortality in IAV-infected mice by inducing a lethal hyper-inflammatory response was considered. It was found that KGF augmentation of IAV-induced inflammation was quite modest and consistent with a slightly higher viral burden (FIGS. 1 and 2) induced by the growth factor. Next, we considered that the strong mitogenic effect of KGF might enhance the susceptibility of AECII directly. The effect of KGF on IAV-induced mortality was time- and dose-dependent, maximal at 5 mg/kg KGF. Low- and high-power microscopic analysis of lung sections from IAV-infected mice stained with antibodies to IAV-NP revealed that KGF administration accelerated spread of infection from the airway to the lung parenchyma, producing peribronchiolar rosettes of IAV-NP positivity corresponding to secondary lobules by 48 h and much more intense and diffuse overall staining by 120 h. The timing of i.t. KGF administration relative to i.t. IAV challenge had a significant effect on AECII infection. The numbers of AECII infected and the mean fluorescence intensity corresponding to the burden of IAV per cell were both at their greatest when the IAV challenge occurred 48 h after i.t. KGF, which was also the point with greatest AECII proliferation based on Ki67 staining. Both the proliferative effects of KGF and the susceptibility of IAV infection returned to baseline by 120 h, consistent with the observation that there is no enhanced mortality effect when KGF is administered to mice 120 h before IAV challenge (FIG. 1C). To assess the effects of KGF on susceptibility of AECII to IAV infection removed from any influence of the host immune system, we performed experiments in which KGF was delivered in vivo but IAV infection was conducted on isolated AECII ex vivo. Again, it was observed that AECII were most vulnerable when infected 48 h after KGF administration, and that susceptibility returned to baseline by 120 h after KGF exposure. In the cells that were IAV infected 48 h after in vivo KGF administration, the proliferating cells (Ki67 high) had a higher viral burden per cell than the nonproliferating (Ki67 low) cells. Consistent with that finding, the expression of the viral protein M1 in IAV-infected AECII also peaked at 48 h post KGF stimulation, based on Western blot analysis of cell lysates from AECII harvested at multiple points after KGF administration and infected ex vivo.

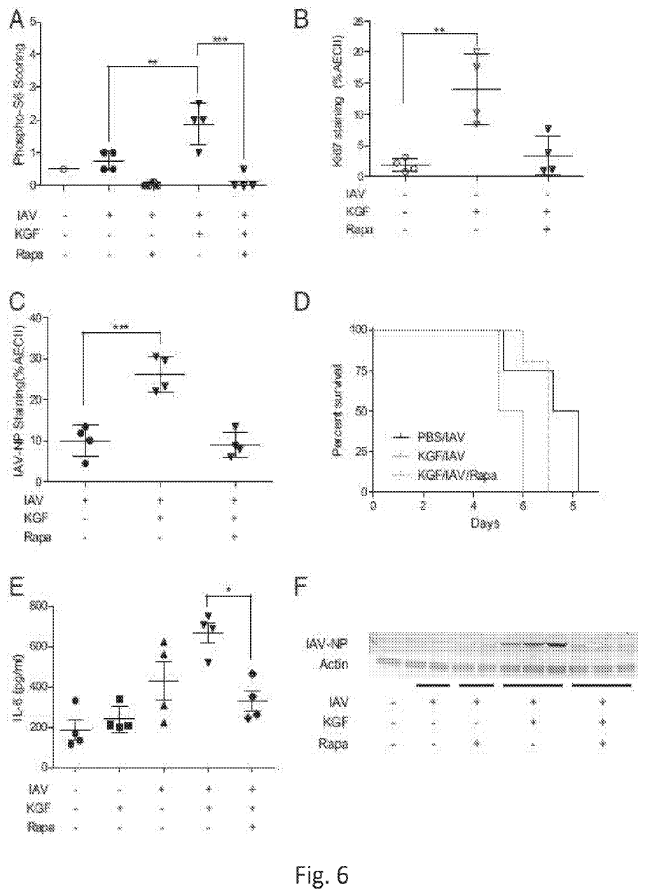

The data support that the molecular mechanism or mechanisms of KGF-enhanced mortality and susceptibility of AECII cells involves activation of the PI3K/Akt/mTOR pathway that is known to facilitate endocytosis and viral replication, in that both the AECII proliferative response (FIGS. 6A and B) and burden of IAV infection (FIGS. 6C and F) were diminished by pretreatment with rapamycin. In addition, rapamycin blocked both KGF-enhanced IL-6 production and KGF-enhanced mortality.

Genomewide RNA interference screening has identified the cognate receptor for KGF, FGFR2, as one of 23 proteins required for viral entry and the PI3K/Akt/mTOR, MAPK, and IP-3 PKC signaling pathways as most essential for early-stage IAV replication. There is also evidence that IAV uses molecular mimicry and other mechanisms to activate cellular signaling pathways to augment its own internalization and proliferation. Binding and clustering of the epidermal growth factor receptor by IAV results in autophosphorylation of the receptor. This signaling event enhances viral internalization by a PI3K/mTOR/AKT pathway, possibly driven by a Trojan horse-like mechanism in which IAV is towed into the cell while bound to epidermal growth factor receptor glycans. In addition, it is known that the NS1 protein produced by IAV binds to the p85β subunit of PI3K to induce PI3K activity in infected cells. Finally, pharmacological intervention with inhibitors of PI3K and rapamycin block IAV replication in vitro, which is consistent with a role for the PI3K/Akt/mTOR pathway in IAV infection.

The data demonstrate a correlation between increased susceptibility to influenza infection and increased proliferation in AECII. The data suggests that the remarkably quiescent state of the AECII in the healthy host, in which less than 1% of AECII are proliferating at any given time, may function as a passive host defense strategy to limit the intrapulmonary spread of IAV infection. The perspective that AECII infection is a pivotal event that determines outcome in influenza infection and that proliferation-dependent AECII susceptibility may be modulated by clinical states such as lung injury raises interesting and potentially clinically relevant questions about our approach to the care of the IAV-infected patient. For instance Sawicki et al. demonstrated almost 50 years ago that exposure of mice to 50% oxygen markedly accelerates IAV- and influenza B virus-induced mortality (Sawicki, et al., Influence of increased oxygenation on influenza virus infection in mice, Lancet 2:680-68 (1961)). Cavalier use of excessive levels of supplemental oxygen in IAV-infected patients may enhance AECII proliferation (as it is known to do in rodents) and promote progression to viral pneumonia.

Other host susceptibility states that are associated with influenza mortality are also of interest. It is known that the fraction of AECII that are proliferating is much higher in infants, which could potentially explain their increased susceptibility to IAV infection. Similarly, cigarette smoking is known to enhance susceptibility to IAV infection in humans, perhaps through increasing pulmonary epithelial cell proliferation, as has been reported in rats. The enhanced mortality of influenza in pregnancy could also be related to hormonal influences on the mitogenic tone of AECII cells, although human or animal data to support this notion is not currently available. Finally, patients with hematologic cancer treated with KGF for stomatitis may be at enhanced risk for lethal consequences from exposure to IAV or other viruses. Consistent with this notion, KGF treatment has been reported to worsen infection caused by anorectal papillomavirus.

These data support that mitogenic stimulation by the lung pulmonary epithelial mitogen KGF hastens spread of influenza to the alveolar epithelium and the subsequent development of lethal influenza pneumonia in mice. The elevated mitogenic tone of the lung may be an overlooked host risk factor that can be exploited by IAV and that should be considered in relation to the enhanced risk for poor outcomes often seen in vulnerable groups.

Therapeutic implications from this line of investigation include more judicious use of supplemental oxygen in patients suspected to have IAV, careful attention of IAV vaccination status in patients who may require KGF treatment for mucositis, and the use of short-course antiproliferative therapies targeting epithelial mTOR activation in patients diagnosed with IAV infection or suspected of having IAV infection, particularly those patients with early and/or rapidly progressive IAV pneumonia.

### Methods of Treatment

Accordingly, provided herein is a method of reducing the risk of influenza A virus infection progressing to viral pneumonia in a patient diagnosed with or suspected of having influenza A virus infection, the method comprising administering to the patient an effective amount of an inhibitor of mammalian target of rapamycin (“mTOR inhibitor”).

In embodiments, the mTOR inhibitor is administered to the patient orally or via inhalation. The mTOR inhibitor may be administered orally in a dosage form selected from the group consisting of a tablet and a liquid suspension or dispersion. Various oral dosage forms of mTOR inhibitors are known in the art. See, for example, WO 97/03654; WO 06/039237; WO 06/094507; U.S. Pat. Nos. 5,989,591; and 8,053,444; each of which is incorporated by reference herein.

In a certain embodiments, the mTOR inhibitor is administered to the patient via inhalation as a pharmaceutical aerosol, for example, via a metered dose inhaler or nebulizer that dispenses a liquid suspension or dispersion comprising the mTOR inhibitor; or via a dry powder inhaler that dispenses the mTOR inhibitor as dry particles. Examples of aerosol formulations are found, for example, in U.S. Pat. No. 5,635,161; exemplary dry powder inhaler formulations are disclosed, for example, in U.S. Pat. No. 9,387,169 and U.S. Pub. 2015/0265582, each of which is incorporated by reference herein.

Various mTOR inhibitors are suitable for use in the present methods. Suitable mTOR inhibitors include, but are not limited to, one or more of rapamycin, everolimus, temsirolimus, deforolimus, zotarolimus, biolimus (umirolimus), and others disclosed, for example, in U.S. Pub. No. 20120064134 and U.S. Pat. No. 9,737,514, each of which is incorporated by reference in its entirety. In a specific embodiment, an mTOR inhibitor is selected from the group consisting of rapamycin, everolimus, temsirolimus, deforolimus, and combinations thereof. In another specific embodiment, the mTOR inhibitor is rapamycin.

The effective dosage of any specific mTOR inhibitor, the use of which is within the scope of embodiments described herein, will vary somewhat from compound to compound and subject to subject and will depend upon the condition of the subject and the route of delivery. The frequency of the administration is generally once or twice per day for a period of from about 3 to about 5 days, or until the condition is essentially controlled and/or therapeutic goal is reached. Lower doses given less frequently can be used prophylactically to prevent or reduce the incidence of recurrence of the infection. In some embodiments, an oral dosage of from about 0.5 to about 2 mg/day will have therapeutic efficacy, with all weights being calculated based upon the weight of the active compound, including the cases where a salt is employed. Typically, a dosage of from about 10 μg to about 200 μg/day can be employed for pulmonary delivery via inhalation.

In certain embodiments, a course of treatment ranges from about 1 day to about 2, 3, 4, 5, 6, 7, 8, 9, or 10 days; from about 2 days to about 3, 4, 5, 6, 7, 8, 9, or 10 days; from about 3 days to about 4, 5, 6, 7, 8, 9, or 10 days; from about 4 days to about 5, 6, 7, 8, 9, or 10 days; from about 5 days to about 6, 7, 8, 9, or 10 days; from about 6 days to about 7, 8, 9, or 10 days; from about 7 days to about 8, 9, or 10 days; from about 8 days to about 9 or 10 days; or from about 9 days to about 10 days.

It has been shown that delivery of the mTOR inhibitor rapamycin to the lungs via inhalation advantageously permits use of a lower dose of rapamycin to achieve therapeutic effect in the lungs, thereby providing lower systemic exposure to the drug and an improved therapeutic index. An aerosol formulation may be delivered to a subject in different ways, for example nasally or perorally, e.g., by inhalation.

In another embodiment, a method of reducing severity of influenza A virus infection is provided, the method comprising administering to a patient suffering from influenza A virus infection an effective amount of a pharmaceutical aerosol comprising an mTOR inhibitor. In some embodiments, reduction in severity is characterized by a reduction in the need for supplemental oxygen, and/or improved dyspnea.

As noted above, various mTOR inhibitors are suitable for use in the present methods. Suitable mTOR inhibitors include, but are not limited to, one or more of rapamycin, everolimus, temsirolimus, deforolimus, zotarolimus, biolimus (umirolimus), and others disclosed, for example, in U.S. Pub. No. 20120064134 and U.S. Pat. No. 9,737,514, each of which is incorporated by reference in its entirety. In a specific embodiment, an mTOR inhibitor is selected from the group consisting of rapamycin, everolimus, temsirolimus, deforolimus, and combinations thereof. In another specific embodiment, the mTOR inhibitor is rapamycin.

In a certain embodiments, the mTOR inhibitor is administered via inhalation as a pharmaceutical aerosol, for example, via a metered dose inhaler or a nebulizer that dispenses a liquid suspension or dispersion comprising the mTOR inhibitor; or via a dry powder inhaler that dispenses the mTOR inhibitor as dry particles.

The effective dosage of any specific mTOR inhibitor, the use of which is within the scope of embodiments described herein, will vary somewhat from compound to compound and subject to subject and will depend upon the condition of the subject and the route of delivery. The frequency of the administration is generally once or twice per day for a period of from about 3 to about 5 days, or until the condition is essentially controlled and/or therapeutic goal is reached. Lower doses given less frequently can be used prophylactically to prevent or reduce the incidence of recurrence of the infection.

In certain embodiments, a course of treatment ranges from about 1 day to about 2, 3, 4, 5, 6, 7, 8, 9, or 10 days; from about 2 days to about 3, 4, 5, 6, 7, 8, 9, or 10 days; from about 3 days to about 4, 5, 6, 7, 8, 9, or 10 days; from about 4 days to about 5, 6, 7, 8, 9, or 10 days; from about 5 days to about 6, 7, 8, 9, or 10 days; from about 6 days to about 7, 8, 9, or 10 days; from about 7 days to about 8, 9, or 10 days; from about 8 days to about 9 or 10 days; or from about 9 days to about 10 days.

In another embodiment, a method of treating a patient diagnosed with or suspected of having influenza A virus infection is provided, the method comprising administering to the patient via inhalation an effective amount of rapamycin. In aspects, the administering reduces risk of the patient developing viral pneumonia. In some aspects, the pharmaceutical aerosol is administered to the patient once or twice daily. In some aspects, the pharmaceutical aerosol is administered to the patient for a period of from about 3 days to about 5 days. In a certain embodiments, the rapamycin is administered via inhalation as a pharmaceutical aerosol, for example, via a metered dose inhaler that dispenses a liquid suspension or dispersion comprising rapamycin; or via a dry powder inhaler that dispenses rapamycin particles as a dry powder.

The effective dosage of rapamycin, the use of which is within the scope of embodiments described herein, will vary somewhat from compound to compound and subject to subject and will depend upon the condition of the subject and the route of delivery. The frequency of the administration is generally once or twice per day for a period of from about 3 to about 5 days, or until the condition is essentially controlled and/or therapeutic goal is reached. Lower doses given less frequently can be used prophylactically to prevent or reduce the incidence of recurrence of the infection.

In certain embodiments, a course of treatment ranges from about 1 day to about 2, 3, 4, 5, 6, 7, 8, 9, or 10 days; from about 2 days to about 3, 4, 5, 6, 7, 8, 9, or 10 days; from about 3 days to about 4, 5, 6, 7, 8, 9, or 10 days; from about 4 days to about 5, 6, 7, 8, 9, or 10 days; from about 5 days to about 6, 7, 8, 9, or 10 days; from about 6 days to about 7, 8, 9, or 10 days; from about 7 days to about 8, 9, or 10 days; from about 8 days to about 9 or 10 days; or from about 9 days to about 10 days.

It will be appreciated that therapeutic benefits for the methods disclosed herein can be realized by combining treatment with an mTOR inhibitor with an antiviral drug. The choice of such combinations will depend on various factors including, but not limited to, the strain of influenza, the age and general health of the patient, the aggressiveness of the influenza infection, and the ability of the patient to tolerate the agents that comprise the combination.

A variety of antiviral agents are suitable for use in combination with the mTOR inhibitors in the methods disclosed herein and include, but are not limited to, amantadine, rimantidine, flumadine, zanamivir, oseltamivir, and combinations thereof.

In some embodiments, the mTOR inhibitor is co-administered with the anti-viral agent. In certain embodiments, “co-administered” means an mTOR inhibitor is administered together with an anti-viral agent in the same unit dosage. In other embodiments, “co-administered” means an mTOR inhibitor and an anti-viral agent are administered in separate dosage forms, concurrently or consecutively.

In additional embodiments, methods are provided herein for the treatment of a patient suffering from an mTOR dependent and/or mTOR-associated viral infection, the method comprising administering to the patient an effective amount of an mTOR inhibitor. In certain embodiments, the viral infection is caused by a pathogen selected from the group consisting of influenza A virus, influenza B virus, adenovirus, rhinovirus, enterovirus, respiratory syncytial virus (RSV), cytomegalovirus (CMV), metapneumovirus (MPV), parainfluenza virus, and the like.

### EXAMPLES

The following detailed methodology and materials are set forth to support and illustrate particular aspects and embodiments of the invention, and should not be construed as limiting the scope thereof.

### Example 1. Materials and Methods

Mouse Models of IAV Infection

Animal studies were conducted with 10-16-wk-old female DBA/2J mice (The Jackson Laboratory). Mice were lightly anesthetized with isoflurane and i.t. inoculated (via the oropharyngeal route) with the SP-D-sensitive hybrid IAV strain, WSNHAnc-Asp225Gly (hereafter referred to as IAV), grown in Madin-Darby canine kidney cells, together with 5 mg/kg KGF in 50 μL Dulbecco's PBS. Animals were monitored daily for vital status. For some experiments, lungs were harvested at 24, 48, and 72 h postviral infection, and the tissues were homogenized in Dulbecco's PBS without Ca²⁺ and Mg²⁺. After centrifugation at 3,000×g for 10 min, the clarified supernatant was collected, aliquoted, and stored at −80° C. In other studies, AECII were isolated 0, 24, 48, or 120 h after i.t. administration of KGF (5 mg/kg), KGF/IAV, or PBS. All animals were maintained in a specific pathogen-free facility and were handled according to a protocol approved by the University of Cincinnati Institutional Animal Care and Use Committee.

Quantification of Viral Particles

Total RNA was isolated from mouse lung homogenates using the QIAamp Viral RNA Mini Kit (Qiagen). Viral RNA was purified by the Viral RNA Spin Protocol (Qiagen), using the Bio-Rad One-Step RT-PCR enzyme mix (Bio-Rad), and the Influenza-A Virus Detection Kit (AD502, iGentBio).

Histological Assessment of Viral Infection

The trachea was cannulated and lungs were inflation-fixed with neutral-buffered 10% formalin (Fisher Scientific) at a pressure of 25 cm H₂O. The lungs were harvested and immersed in 10% formalin overnight, embedded in paraffin, cut into 5-μm sections, and mounted on Trubond 380 adhesive microscope slides (Tru Scientific). Endogenous peroxidase activity was quenched with 0.3% hydrogen peroxide in methanol; sections were blocked with 15% normal goat serum and incubated with rabbit anti-IAV NP antibody (Virostat; 1:1,500 dilution) overnight at 4° C. Slides were developed by incubation with biotin-labeled goat anti-rabbit IgG secondary antibody (1:750 dilution) and avidin-peroxidase-dependent (Vector Laboratories) oxidation of diaminobenzidine. A blinded analysis of the burden of viral infection in conducting airways and lung parenchyma was performed by scoring the intensity of immunohistochemical staining for IAV-NP, using a semiquantitative scale [i.e., −1 (least) to 4 (most)].

Cytokine Determination

Inflammatory cytokines in clarified supernatants of whole-lung homogenates were measured by ELISA, according to the manufacturer's instructions (R&D Systems). Multianalyte studies of BAL fluid and serum were conducted by ELISA, using Milliplex Multiplex kits (Millipore) according to the manufacturer's protocol. Concentrations were calculated from standard curves, using recombinant protein standards and expressed in picograms per milliliter.

Preparation of Dispersed Cells from Whole Lung

Explanted lungs were instilled with 5,000 caseinolytic units (c.u.)/mL dispase (BD Biosciences) via the i.t. route and then immersed (Worthington Biochemicals) in 3 mL ice cold dispase (5,000 c.u.)/mL containing 50 U/mL DNase. Lung tissues were then processed in a gentleMACS Dissociator according to manufacturer's instructions (Miltenyi Biotec), and cell suspensions were passed through a 40-μm filter before washing with Dulbecco's PBS and subsequent analysis.

Flow Cytometry and Identification of Epithelial Cell Subtypes

Fixation of dispersed lung cells was accomplished either in sequential steps, using ammonium chloride lysis solution (Stemcell Technologies) followed by paraformaldehyde (1.6% for 10 min), or in a single step, using eBioscience fix/lysis reagent. Cells were washed and resuspended in 70% methanol/FACS buffer overnight before washing and incubation with antibodies. AECII and pulmonary epithelial cells were identified by staining with anti-pro SPC (Seven Hills Bioreagents) and anti-CD326 (clone G8.8; eBioscience), respectively. Alveolar macrophages were identified using antibodies to F4/80 (clone BM8; eBioscience) and CD11c (clone N418; eBioscience). Influenza virus was detected using an antibody to IAV-NP (clone D67J; Thermo Fisher Pierce). Proliferative status was determined using the Ki67 antibody (clone SOLA15; eBioscience). Compensation for fluorescence overlap and background fluorescence was accomplished using fluorescence minus one controls. Samples were run on a FACS LSRII, using four lasers (325, 405,488, and 633 nm) (Becton Dickenson). Data were analyzed using FCS Express flow cytometry software.

Isolation, Enrichment, and IAV Infection of AECII

Ex vivo IAV infectivity assays were performed with AECII isolated as previously described (Wu, et. al., Keratinocyte growth factor augments pulmonary innate immunity through epithelium-driven, GM-CSF-dependent paracrine activation of alveolar macrophages, J Biol Chem 286:14932-40 (2011) 0, 24, 48, and 120 h after i.t. administration of PBS or 5 mg/kg KGF. Briefly, saline-perfused lungs were instilled with 5,000 c.u. dispase via the i.t. route, followed by 0.5 mL melted agarose. The lungs were immersed in a 3-mL solution containing 5,000 c.u./mL dispase, incubated at 37° C. for 45 min, and gently teased apart in RPMI media containing DNase. The cell suspension was filtered through 40 μm membranes and panned on plates coated with anti-CD45 and CD16/32 (BD Biosciences) to remove immune cells and fibroblasts. After RBC lysis, an aliquot of the cell suspension was spun onto slides (Thermo-Shandon) and stained with pro-SPC antibody and Trypan blue to determine purity and viability of AECII, respectively. Cell suspensions that were verified to be at least 85% pure AECII were then plated in 24-well plates at a density of 2.5×10⁵ cells/well, and incubated with 2.5×10⁴ IAV particles in media (RPMI with 5% FBS) or media alone for 18 h at 37° C. in a 5% CO₂ atmosphere. Cells were harvested, washed, fixed, permeabilized, and stained with antibodies before analysis by flow cytometry, as described earlier. For Western blot analysis, cell lysates were prepared in RIPA lysis buffer with protease and phosphatase inhibitors. Equal amounts of clarified supernatants were size-fractionated on 4-20% SDS/PAGE gels and transferred to nitrocellulose membranes. The blots were probed with IAV-M1 antibody (ABD-Serotec), followed by secondary antibodies conjugated with IRDye 800CW linked to goat anti-rabbit or goat anti-mouse (Li-Cor). GAPDH was used as a loading control. Images were captured using a Li-Cor Odyssey per manufacturer's instructions.

Isolation, Primary Culture, and Infection of Rat AECII in Vitro

In vitro infection assays were performed on rat AECII isolated from specific-pathogen-free Sprague-Dawley rats (150-200 g) (Charles River). Rats were killed with Euthasol and lungs were perfused via the pulmonary artery with sterile pulmonary perfusion buffer (140 mM sodium chloride, 2.5 mM sodium phosphate, 10 mM Hepes, 5 mM potassium chloride, 6 mM d-glucose, 2 mM calcium chloride, 1.3 mM magnesium sulfate) until they blanched. The lungs were lavaged through the trachea with 8 mL pulmonary lavage buffer (140 mM sodium chloride, 2.5 mM sodium phosphate, 10 mM Hepes, 5 mM potassium chloride, 6 mM d-glucose) five times to remove inflammatory cells. Lungs were excised and filled with 3 U/mL elastase (Worthington) in pulmonary perfusion buffer and incubated at 37° C. for 20 min. The pulmonary tissue was finely minced with scissors and immersed in 10 mL pulmonary perfusion buffer containing 4,000 U DNase-I. Lungs were gently swirled for 3 min at 37° C. to dissociate cells from the digested tissue. The cells were filtered sequentially through sterile gauze and 100- and 20-μm Cell Strainers (Fisher Scientific), and pelleted for 5 min at 400×g. The cell pellet was resuspended with red blood cell lysis buffer for 1 min and pelleted for 5 min at 400×g. Cells were resuspended in 10 mL DMEM-Hepes without FBS and incubated for 1 h in tissue culture plates coated with rat IgG at 37° C. with 5% CO₂. Nonadherent cells were collected and suspended in 10 mL DMEM with high glucose and 10% FBS. The purity of AECII cells was determined by the staining of lamellar bodies with lysotracker (Invitrogen) and examining the cells under high magnification. The AECII (4×10⁵ cells/well) were plated in 24-well tissue culture plates coated with collagen IV (Corning Biocoat) in 0.5 mL DMEM with high glucose and 10% FBS, at 37° C. with 5% CO₂. AECII were cultured until 60-80% confluence was observed (˜24 h) and then serum starved in DMEM for 18 h. Cells were incubated with the signaling inhibitors 2.5 μM BGJ398 (Selleckchem) or 40 nM rapamycin (LC Laboratories) 30 min before incubation with IAV in the presence or absence of KGF (50 ng/mL) for 60 min at 37° C. Cells were harvested and lysed in RIPA buffer containing phosphatase and protease inhibitors at 15 and 60 min after addition of IAV and KGF. Viral protein loads were determined by Western blot 18 h after IAV infection. Equal protein loads were run on 4-20% SDS/PAGE gels, transferred to nitrocellulose membranes, and probed with rabbit anti-influenza A NP antibody, 1:250 (Thermo Fisher); mouse anti-influenza A matrix protein 1, 1:250 (Bio-Rad); and mouse anti-β-actin antibody 1:20,000 (Sigma). IRDye 800CW goat anti-mouse IgG and goat anti-rabbit IgG were used as secondary antibodies (1:10,000). The blots were developed using the LI-COR Odyssey imaging system.

### Example 2. Intrapulmonary Administration of KGF Enhances Mortality and Viral Burden after Pulmonary IAV Challenge

Experiments were conducted to test the hypothesis that KGF administration would enhance survival of mice challenged with WSNHAnc-Asp225Gly (a SP-D-sensitive, hybrid IAV strain, WSNHAnc-Asp225Gly, hereafter referred to as IAV), and the results are shown in FIG. 1A. Animals were inoculated via the intratracheal (i.t.) route with a PBS solution containing 1,500 IAV particles with or without 5 mg/kg KGF, and vital status was followed over the course of 11 d. Although all animals ultimately succumbed to IAV infection, KGF coadministration shortened survival by an average of 3 d (P=0.01). Coadministration of IAV with KGF at concentrations spanning 4 logs (0.1, 1, 10, and 100 μg/mouse) resulted in dose-dependent acceleration of mortality (FIG. 1B). Pretreatment with KGF 120 h before inoculation with IAV did not enhance mortality compared with mice given PBS/IAV at time 0 (FIG. 1C). Mice treated with concomitant i.t. IAV and KGF (5 mg/kg) had a three- to fivefold greater pulmonary viral burden by 48 and 72 h postchallenge compared with PBS/IAV-treated controls, based on real-time PCR of IAV RNA in whole-lung homogenates (FIG. 1D; P<0.01). No difference in viral burden was detected between the PBS/IAV and KGF/IAV groups at the 24 h postinoculation point.

### Example 3. Effect of KGF and IAV on Inflammatory Profiles in BALF, Serum, and Whole-Lung Homogenates

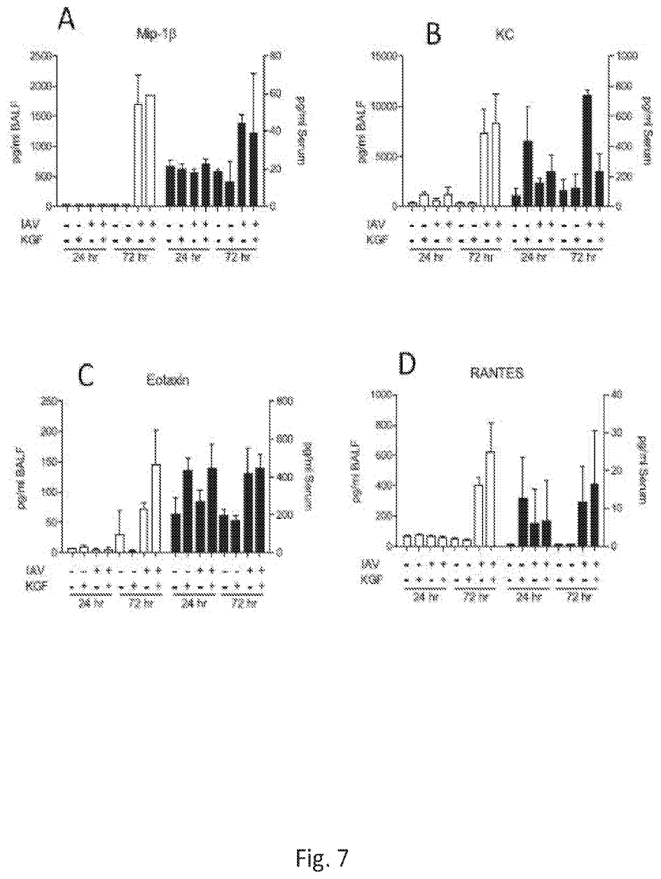

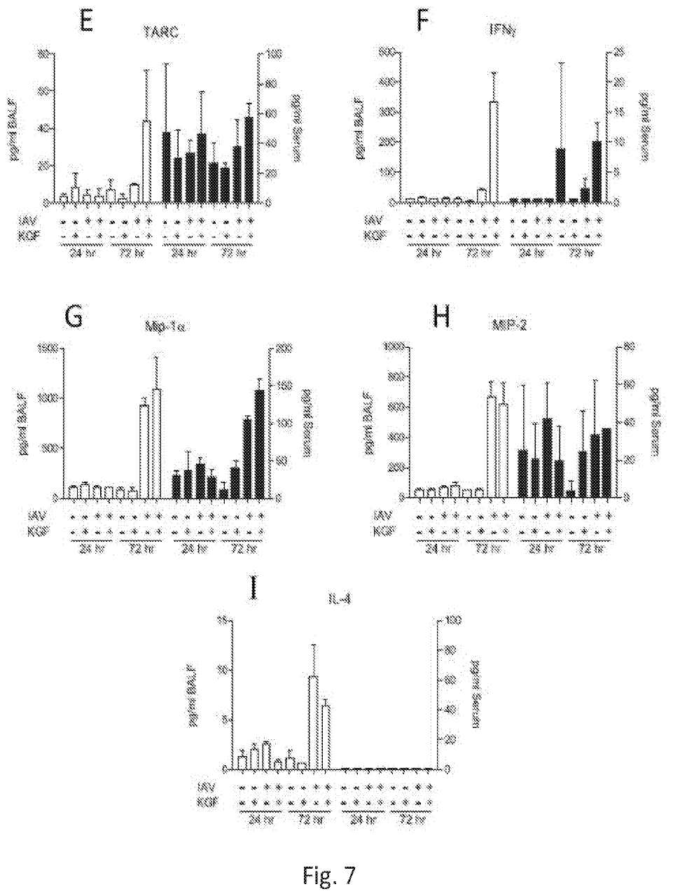

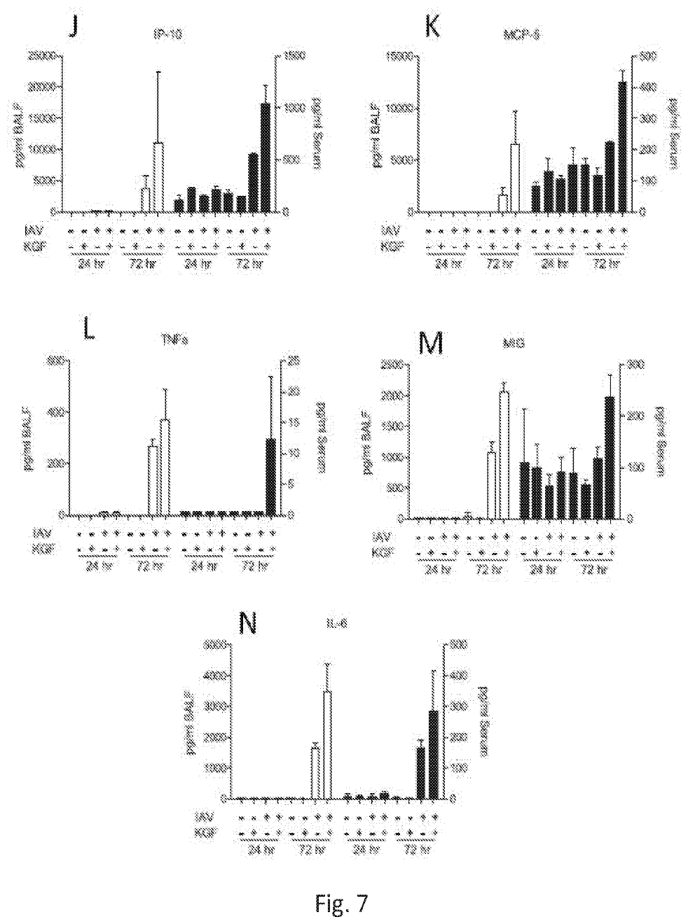

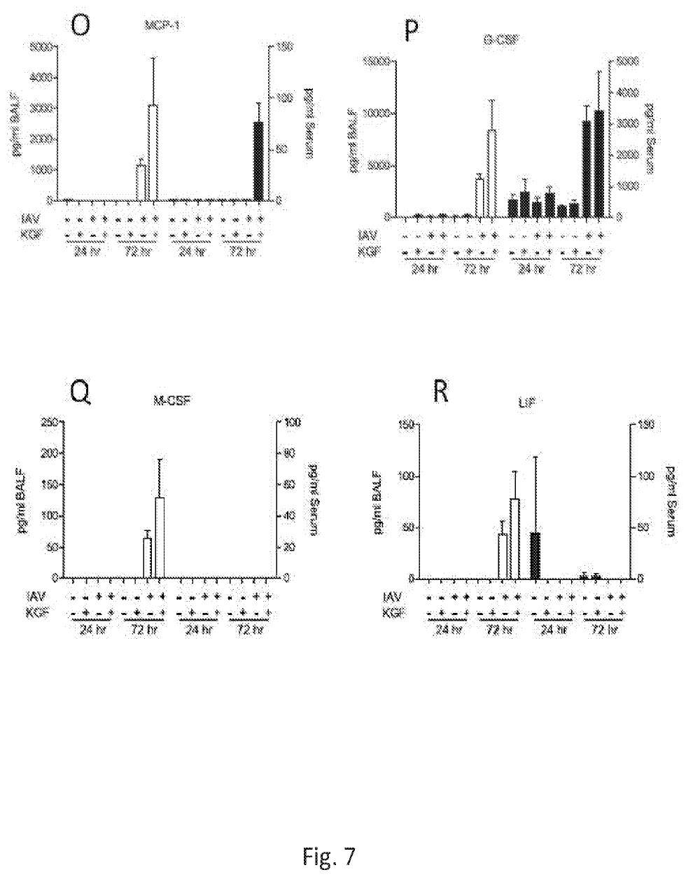

To explore the role of inflammation in the KGF-augmented mortality of IAV-infected mice, a multiplex analysis was conducted of inflammatory mediators in serum and acellular broncho-alveolar lavage fluid (BALF) collected at 24 and 72 h after i.t. challenge with 1,500 viral particles of IAV in PBS with or without 5 mg/kg KGF (FIGS. 2A and B and FIG. 7A-R). Changes in selected cytokines at 72 h postchallenge are shown as fold change from PBS controls. Coadministration of KGF with IAV modestly augmented BAL IP10, MCP-5, TNFα, MIG, IL-6, MCP-1, G-CSF, M-CSF, and IFNγ responses relative to the PBS/IAV control group. KGF also augmented the inflammatory response to IAV challenge in serum for some cytokines, especially for IP10, TNFα, MCP-1, and IFNγ. To confirm changes to pulmonary proinflammatory cytokines, levels of IL-6, TNFα, KC, and MCP-1 were also measured by ELISA in whole-lung homogenates at 24, 48, and 72 h after IAV challenge in the presence or absence of KGF (FIG. 2 C-F). IAV alone produced three- to eightfold increases in IL-6, TNFα, KC, and MCP-1 levels at 48 and 72 h, which were modestly enhanced (by ˜10-50%) by KGF coadministration. Control experiments were also performed to determine the effect of KGF treatment alone. There was no effect of KGF treatment alone on cytokine responses at any point, except for two- to threefold increases in TNFα at 48 and 72 h postchallenge (FIG. 2D) compared with naive controls.

### Example 4. KGF Promotes the Spread of Viral Infection from the Airways to the Lung Parenchyma

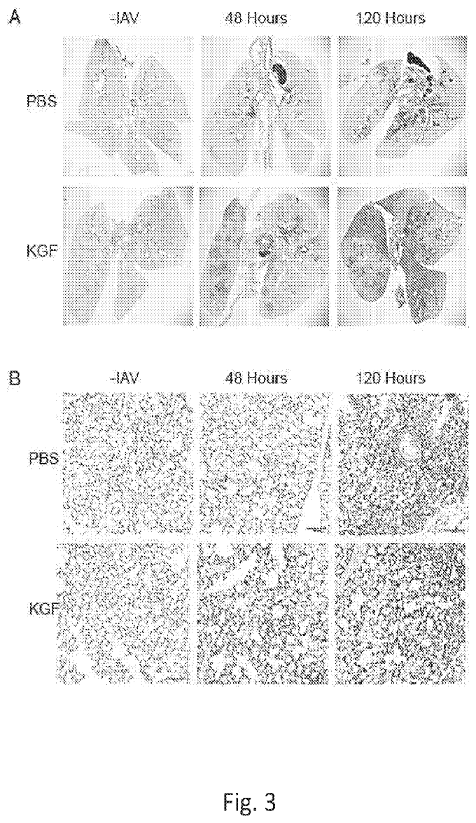

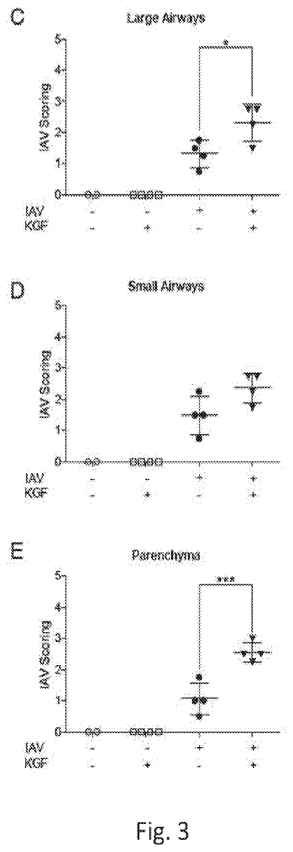

Experiments were conducted to determine the effects of KGF on the spatial and temporal pattern of pulmonary IAV infection. Influenza nucleoprotein (IAV-NP) staining was performed on inflation-fixed whole-lung sections (FIGS. 3A and B) at 48 and 120 h after i.t. inoculation of 1,500 viral particles of IAV, with and without KGF coadministration. At 48 h after viral challenge, IAV-NP staining was faint and centered on large airways in mice that received only PBS with IAV. At the same time, in IAV-challenged mice given i.t. KGF, staining was substantially more intense, and extensive peribronchiolar spread into the surrounding lung parenchyma was readily apparent on both low-power (FIG. 3A) and high-power (FIG. 3B) views. By 120 h, IAV-NP staining was increased in both groups, but was markedly more intense and diffuse in the KGF group. Blinded semiquantitative analysis of IAV-NP staining in high-power lung sections was consistent with a KGF-induced increase in viral burden and acceleration of spread from conducting airways to the lung parenchyma (FIG. 3C-E).

### Example 5. KGF-Enhanced AECII Infectivity Correlates with Their Proliferative State, in Vivo

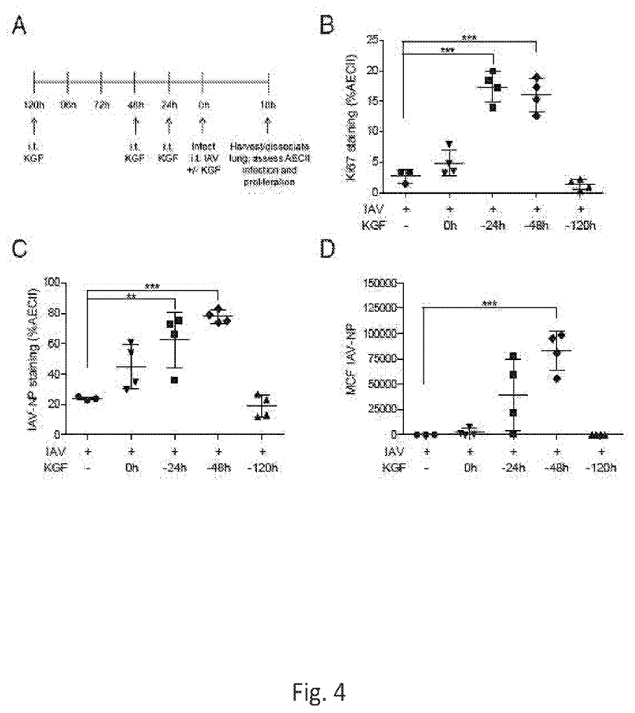

Experiments were conducted to determine whether the mitogenic effects of KGF enhance the susceptibility of AECII to IAV infection in vivo. Single-cell suspensions were prepared from whole-lung homogenates 24 h after high-dose (50,000 viral particles) i.t. IAV challenge of mice that had been pretreated with i.t. KGF at 24, 48, or 120 h before infection (FIG. 4A). The dispersed cells were then washed, fixed, and stained with anti IAV-NP to detect IAV infection, anti-pro SP-C to identify AECII, and anti-Ki67, a marker of cellular proliferation. The percentage of AECII that were proliferating according to Ki67 staining increased in a time-dependent manner, peaking in cells isolated from mice that had been dosed with KGF 48 h before PBS challenge and remaining similar to PBS control levels in those given KGF 120 h before PBS challenge (FIG. 4B). The percentage of AECII that were infected (based on IAV-NP staining) also peaked in dispersed lung cells from mice that had been pretreated with KGF 48 h before infection and remained near PBS control levels in mice given KGF 120 h before delivery of the inoculum (FIG. 4C). The mean channel fluorescence (MCF), reflecting viral burden per cell, followed a similar pattern, peaking in cells harvested at 48 h after KGF and remaining close to PBS control levels in cells harvested at 120 h post KGF (FIG. 4D). The correlation between peak AECII proliferation and peak AECII infectivity suggests mitogenically stimulated cells are more susceptible to infection.

### Example 6. KGF Produces a Time-Dependent Increase in Ex Vivo IAV Infection of AECII that Correlates with their Proliferative Tone

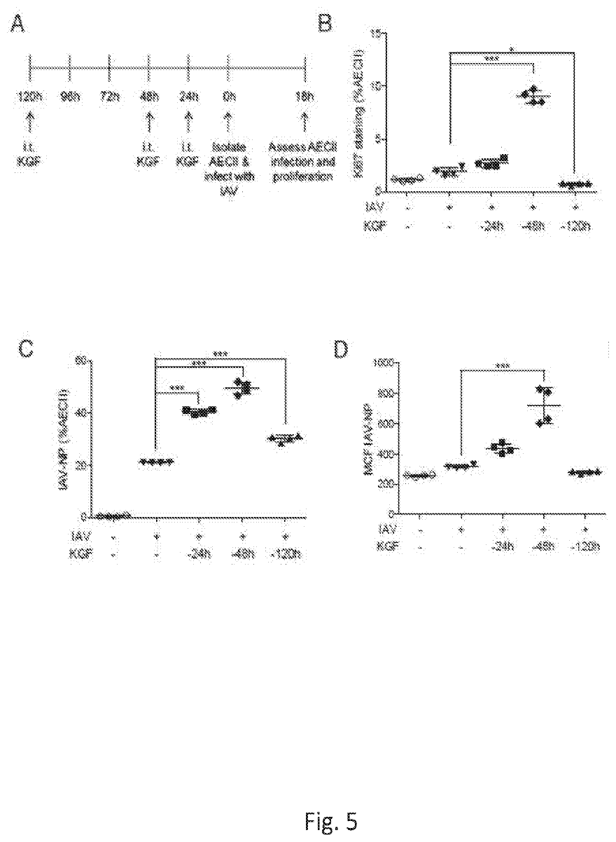

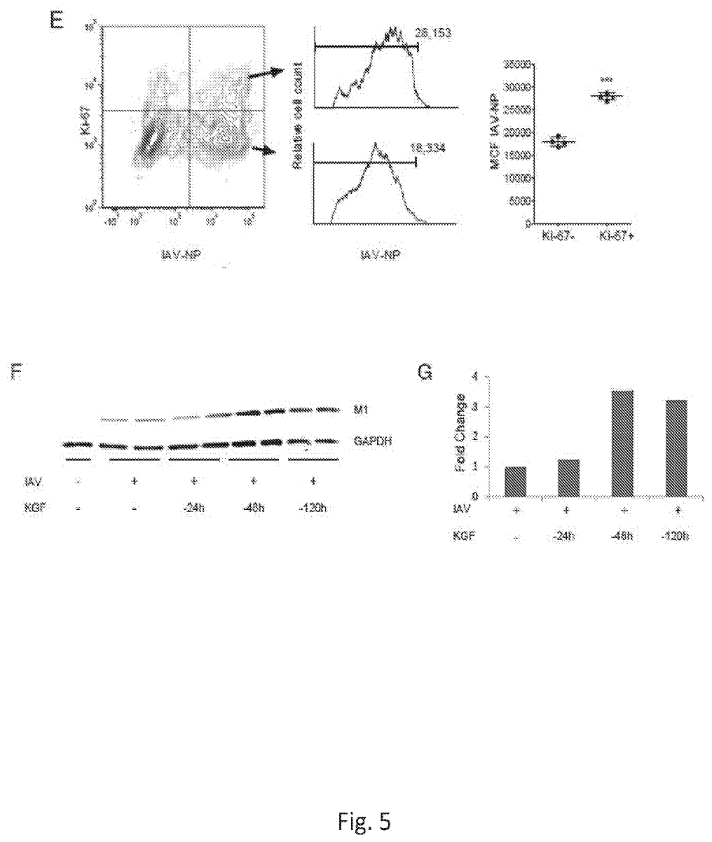

Ex vivo experiments were conducted to examine the role of the mitogenic effects of KGF on the cell autonomous susceptibility of AECII to IAV infection. AECII were isolated from mice that had been treated with i.t. KGF at 24, 48, or 120 h before and cultured ex vivo with IAV at a multiplicity of infection of 10 in media or media alone (RPMI with 5% FBS) for 18 h (FIG. 5A). The cells were then washed, fixed, and stained with anti-IAV-NP, anti-pro SP-C, and anti-Ki67. In AECII isolated at various points after KGF administration, the percentage of AECII that were proliferating based on Ki67 staining rose at 24 h, peaked at 48 h, and returned to near baseline by 120 h post KGF (FIG. 5B). The percentage of AECII that were infected after ex vivo IAV incubation was also highest in cells harvested 48 h after in vivo KGF treatment and remained near baseline levels in the cells harvested at 120 h post KGF (FIG. 5C). The MCF per cell for IAV-NP followed a similar pattern, peaking in cells harvested at 48 h post KGF and returning to baseline in cells harvested at 120 h after KGF (FIG. 5D). Although these analyses revealed an increased percentage of infected AECII and an increased viral burden per AECII (based on mean channel fluorescence) that was associated with the peak in AECII proliferation, these data are correlative and an additional ex vivo experiment was conducted to better establish a causal relationship between proliferation and infectious susceptibility. In AECII harvested 48 h after in vivo KGF administration and infected with IAV ex vivo, the mean fluorescence intensity of IAV-NP staining was greater in the Ki67 high (proliferating) than the Ki67 low (nonproliferating) population, consistent with preferential infection or viral replication in cycling cells (FIG. 5E). The peak in expression of viral protein M1 in the lysates of isolated AECII infected with IAV ex vivo also occurred in AECII that were isolated 48 h after in vivo KGF challenge, consistent with greater susceptibility of cycling AECII to IAV infection compared with quiescent cells (FIGS. 5F and G).

### Example 7. Rapamycin Inhibits KGF-Induced Increases in Mortality, Proliferation, and Viral Susceptibility in Vivo

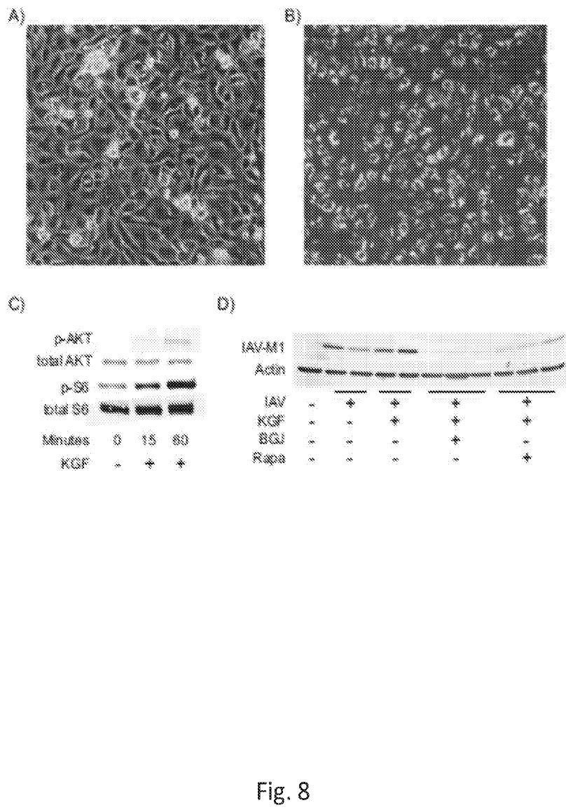

KGF is known to signal through the MAPK and PI3K/Akt/mTOR pathway. Experiments were conducted to determine whether KGF-induced changes in proliferative tone of AECII could be inhibited with the mTOR inhibitor, rapamycin, in vivo. Mice were treated with once-daily i.p. rapamycin (1 mg/kg) or vehicle control for 4 d before i.t. inoculation with PBS, PBS containing KGF (5 mg/kg) or PBS containing KGF with or without IAV. Lungs were inflation-fixed with formalin at 24 h after i.t. challenge, sectioned and stained with an antibody directed against ribosomal S6 protein phosphorylated at position Ser-235/236. Blinded semiquantitative immunohistochemical analysis revealed a rapamycin-inhibitable increase in phospho-S6 staining of the pulmonary parenchyma in the IAV+KGF group compared with IAV alone, (FIG. 6A). Single-cell suspensions were prepared from whole-lung homogenates 48 h after KGF inoculation with or without rapamycin pretreatment of the animals. The dispersed cells were then washed, fixed, and stained with anti-pro SP-C, anti-phospho-S6, and anti-Ki67. The percentage of AECII that were proliferating based on Ki67 staining increased more than sevenfold from 2% to 14% in the KGF inoculated mice, relative to mice given only PBS. Rapamycin pretreatment for 4 d before KGF challenge blocked the proliferative response to KGF (FIG. 6B). Next, experiments were conducted to determine whether KGF-induced enhancement of AECII infectivity could be inhibited with rapamycin in vivo. Mice were treated with rapamycin (1 mg/kg) for 4 d before i.t. inoculation with IAV±KGF. Single-cell suspensions were prepared from whole-lung homogenates 72 h after infection. The dispersed cells were then washed, fixed, stained with anti-IAV-NP and anti-pro SP-C, and analyzed by flow cytometry. The percentage of AECII that were infected was significantly greater in the KGF-inoculated mice compared with PBS control mice, and the effect was prevented by in vivo pretreatment with rapamycin (FIG. 6C). Experiments were conducted to determine whether the observed rapamycin-mediated reduction in AECII proliferation and infectivity attenuated the severity of IAV infection in vivo. Rapamycin pretreatment resulted in a significant reduction in KGF-accelerated mortality (FIG. 6D; P<0.05), as well as a significant reduction of the KGF-induced increase in the lung tissue levels of the proinflammatory cytokine, IL-6 (FIG. 6E). Western blot analysis of IAV-NP levels in whole-lung homogenates harvested 24 h after in vivo infection indicate that rapamycin attenuates IAV production in vivo (FIG. 6F). To confirm that KGF-induced viral susceptibility was receptor mediated and to rule out off-target effects, the effect of the pan-FGFR inhibitor BGJ398 on KGF-enhanced viral susceptibility was examined. A rat AECII primary culture system was developed that was both KGF-responsive and permissive to infection with the IAV strain used in this study. Both inhibition of FGF receptor with BGJ398 and mTOR inhibition with sirolimus blocked KGF-induced IAV susceptibility (FIGS. 8C and D). These data suggest that enhancement of IAV infection and/or replication by KGF, which is known to signal through multiple pathways including Jak/Stat, MAPK, and PI3K, can be at least partially inhibited by targeted blockade at two points within the FGFR/Akt/PI3K/mTOR pathway.

All documents cited are incorporated herein by reference; the citation of any document is not to be construed as an admission that it is prior art with respect to the present invention.

It is to be further understood that where descriptions of various embodiments use the term “comprising,” and/or “including” those skilled in the art would understand that in some specific instances, an embodiment can be alternatively described using language “consisting essentially of” or “consisting of.”

While particular embodiments of the present invention have been illustrated and described, it would be obvious to one skilled in the art that various other changes and modifications can be made without departing from the spirit and scope of the invention. It is therefore intended to cover in the appended claims all such changes and modifications that are within the scope of this invention.

1. A method of reducing risk of influenza A virus infection progressing to viral pneumonia in a patient diagnosed with or suspected of having influenza A virus infection, the method comprising administering to the patient an effective amount of an inhibitor of mammalian target of rapamycin (mTOR).

2. The method of claim 1, wherein the mTOR inhibitor is administered orally or via inhalation.

3. The method of claim 2, wherein the mTOR inhibitor is administered orally in a dosage form selected from the group consisting of a tablet and a liquid suspension.

4. The method of claim 2, wherein the mTOR inhibitor is administered via inhalation as a pharmaceutical aerosol.

5. The method of claim 4, wherein the pharmaceutical aerosol comprises a dry powder comprising the mTOR inhibitor.

6. The method of claim 4, wherein the pharmaceutical aerosol comprises a liquid suspension comprising the mTOR inhibitor.

7. The method of claim 1, wherein the mTOR inhibitor is selected from the group consisting of rapamycin, everolimus, temsirolimus, ridaforolimus, and combinations thereof.

8. The method of claim 1, wherein the mTOR inhibitor is administered to the patient once or twice daily.

9. The method of claim 8, wherein the mTOR inhibitor is administered to the patient for a period of from about 3 days to about 5 days.

10. A method of reducing severity of influenza A virus infection, the method comprising administering to a patient suffering from influenza A virus infection an effective amount of a pharmaceutical aerosol comprising an mTOR inhibitor.

11. The method of claim 10, wherein the mTOR inhibitor is selected from the group consisting of rapamycin, everolimus, temsirolimus, ridaforolimus, and combinations thereof.

12. The method of claim 10, wherein the pharmaceutical aerosol is administered to the patient once or twice daily.

13. The method of claim 12, wherein the pharmaceutical aerosol is administered to the patient for a period of from about 3 days to about 5 days.

14. The method of claim 10, wherein pharmaceutical aerosol comprises a dry powder comprising the mTOR inhibitor.

15. The method of claim 10, wherein the pharmaceutical aerosol comprises a liquid suspension comprising the mTOR inhibitor.

16. A method of treating a patient diagnosed with or suspected of having influenza A virus infection, the method comprising administering to the patient via inhalation an effective amount of rapamycin, whereby the administering reduces risk of the patient developing viral pneumonia.

17. The method of claim 16, wherein the rapamycin is administered as a pharmaceutical aerosol.

18. The method of claim 17, wherein the pharmaceutical aerosol comprises a dry powder comprising rapamycin particles.

19. The method of claim 16, wherein the rapamycin is administered once or twice daily for a period of from about 3 days to about 5 days.
